(19) United States
(12) Patent Application Publication
(10) Pub. No.: US 2018/0100130 A1
(43) Pub. Date: Apr. 12, 2018

(54) METHOD AND DEVICE FOR STABILIZING POLYPHENOL-CONTAINING LIQUIDS

(71) Applicant: KHS GmbH, Dortmund (DE)

(72) Inventor: Alois Monzel, Mörstadt (DE)

(21) Appl. No.: 15/567,171

(22) PCT Filed: Apr. 19, 2016
(86) PCT No.: PCT/EP2016/058635

§ 371 Date: Oct. 17, 2017

(30) Foreign Application Priority Data
Apr. 23, 2015 (DE) 10 2015 106 258.5

(51) Int. Cl. C12H 1/056; C12H 1/07; C12H 1/16; A23L 2/72; A23L 2/80; G01N 33/14; G01N 33/00; G01T 1/36

(52) U.S. Cl. CPC C12H 1/0424; C12H 1/063; C12H 1/165; A23L 2/72; A23L 2/80; A23V 2300/38; G01N 33/0036; G01T 1/36; A23V 2002/00; A23V 2300/02; G01N 33/146

(57) ABSTRACT

A process for stabilizing a liquid that contains a first type of polyphenol includes measuring the liquid's spectrum, comparing it to a reference spectrum, based on the comparison, estimating an amount of the first type of polyphenol, based on the amount, adding a specified quantity of an insoluble stabilizing agent to the liquid, feeding the stabilizing agent and the liquid to a filter unit, at the filter unit, separating the stabilizer from the liquid, and discharging the liquid. The specified quantity is based on the amount.

### RELATED APPLICATIONS

This application is the national stage, under 35 USC 371, of international application PCT/EP2016/058635, filed on Apr. 19, 2016, which claims the benefit of the Apr. 23, 2015 priority date of German application DE 102015106258.5, the content of which is herein incorporated by reference.

### FIELD OF INVENTION

The invention relates to a method for stabilizing liquids containing at least one type of polyphenol, and a device for carrying out such a method.

### BACKGROUND

Certain substances cause wine or beer to become prone to colloidal turbidity. Among these substances are polyphenols. These are aromatic compounds that are often present in the plants used to make these beverages.

Turbidity tends to occur when proteins in the beverage react with polyphenol compounds. Although the turbidity does not affect storage life, it is aesthetically displeasing for most consumers. It is therefore desirable to inhibit such turbidity.

To suppress turbidity, it is known to remove substances from the liquid that may promote turbidity. This process is called “stabilization.”

### SUMMARY

One way to remove polyphenols from beer is to begin by filtering out yeasts and other substances associated with turbidity. The beer is then first brought in contact with a stabilizing agent, which represents a preferred reaction partner for the polyphenolic compounds and/or specific protein fractions. In some cases, the stabilizing agent is lost. In others, it is regenerated. A suitable stabilizing agent is polyvinylpolypyrrolidone, which bonds to polyphenols. The polyphenols bonded to the polyvinylpolypyrrolidone are then removed from the beer, for example by filtration. The polyvinylpolypyrrolidone can then be regenerated, for example, by exposure to caustic soda.

An alternative is that of using enzymes to remove or substantially reduce the amount of polyphenols from beer. Examples of enzymes that are suitable for this include pepsin, papsin, or Brewer's Clarex.

The degree to which polyphenols can be removed from liquids depends on many factors. Of particular significance is the quantity of stabilizing agent added, the number of free adsorption points of the stabilizing agent, and the length of the contact time of the liquid with the stabilizing agent. By suitable selection and arrangement of the components of a stabilizing device, for example tank volume, flow rates, etc., the entire stabilizing device can be arranged in such a way that adequate contact times can be achieved between the stabilizing agent and the constituents that are to be removed.

The stabilizing effect of a stabilizing agent, such as polyvinylpolypyrrolidone, decreases as it is used. It is therefore useful to add fresh stabilizing agent at approximately the same time as the delivery of the beer that is to be stabilized.

Once the beer reaches the desired degree of stabilization, it is useful to filter the stabilizing agent from the beer. This can carried out by powder or settling filters, or other suitable methods. Suitable filters for powder or settling filtration include horizontal filters and candle filters. In some cases, it is particularly useful to convey the liquid to two filtering units that are operated out of phase so that non-stabilized liquid with stabilizing agent added is conveyed to each filter unit after a regeneration phase and cleaning phase.

A difficulty that arises is that the total quantity of polyphenols in the liquid is difficult to determine in advance. Thus, it is difficult to know how much stabilizing agent should be added.

Since under-stabilization is so undesirable, it is desirable to add stabilizing agent in quantities that ensure stabilization. However, this leads to higher costs.

An object of the invention is to provide a method and a device that promote more economical use of stabilizing agents.

The present invention makes use of the surprising finding that a spectroscopic examination of liquids containing polyphenols in conjunction with an analysis of the emission lines or absorption lines allows one to infer the polyphenol content of the liquid being examined. This is particularly successful if the measured spectrum is compared with reference or comparison spectra. As used herein, the term “spectrum” is to be understood as the result of spectral examination, and in particular, the emission or absorption lines at different wavelengths or wave numbers.

The present invention provides a method for stabilizing liquids that contain at least one kind of polyphenol.

In a first step, a liquid containing polyphenol is supplied by a supplying-and-feeding unit. Then, in a second step, a measuring device measures a spectrum of the liquid. In a third step, a comparison-and-analysis unit estimates the amount of polyphenol contained in the liquid. It does so by comparing the spectrum detected from the second step with a reference spectrum. Based at least in part on this comparison, it estimates for the amount of polyphenol contained in the liquid.

In a fourth step the follows the third step, a metering unit adds insoluble stabilizing agent to the liquid. This stabilizing agent is one that is configured to bind to polyphenol in the liquid. The quantity of stabilizing agent is determined as a function of the estimate from the third step's estimate of the amount of polyphenol in the liquid.

A fifth step includes feeding the mixture of liquid and stabilizing agent to a filter unit. This leads to a sixth step of separating the stabilizing agent, now at least partially loaded with polyphenol, from the liquid. A seventh step that follows includes discharging the liquid through a discharge line. This liquid is now a stabilized liquid thanks to the removal of polyphenol by the stabilizing agent.

A particular advantage is that, by measuring and recording at least one spectrum of the liquid that is to be stabilized using the foregoing method, it becomes possible to estimate a value for the quantity of at least one species of polyphenol contained in the liquid. The quantity of polyphenol contained in the liquid is understood to mean one of: the content or total content of polyphenol in the liquid, the polyphenol substance quantity obtained, and the concentration of polyphenol.

As a result of being able to estimate this quantity, it is possible to add stabilizing agent to the liquid as a function of the quantity of polyphenol. This avoids over-metering and reduces costs.

In some practices, estimating the amount of polyphenol in the liquid comprises comparing at least one spectrum of the liquid with a reference spectrum and evaluating the result of this comparison. A suitable reference spectrum is one obtained from a polyphenol-free sample of the same liquid. Alternatively, a reference spectrum could be that of a liquid that has a non-zero but known polyphenol content.

Polyphenol can be present in different species at the same time, each with its own characteristic spectrum. Therefore, it is advantageous to use a plurality of reference spectra, each of which corresponds to one of the polyphenol-bearing species.

The reference spectrum is particularly well suited for comparison analyses. To carry out the evaluation and resulting estimate of polyphenol content, at least one recorded spectrum from the liquid and at least one reference spectrum are subjected to suitable and known comparison operations, calculation operations, and/or transformations. Through comparative analysis, it is thus possible to estimate the quantity of polyphenol contained in the liquid.

The reference spectrum is preferably recorded before the method is carried out and preferably before making use of the same measuring device that is also used for the method itself.

As an alternative, a reference spectrum can also be recorded during the performance of the method but before the method's third step. This ensures that a current reference spectrum is available for each polyphenol species of a liquid that is to be stabilized.

As an alternative, however, pre-recorded reference spectra can also be provided for liquids that are to be stabilized.

For example, depending on the application, depending on specific provisions and operational rulings or on the judgment of a person skilled in the art, a recorded reference spectrum can be used over a predetermined period of time. After the expiration of the predetermined period of time, a reference spectrum is again recorded for further use.

A group of polyphenols includes a considerable number of polyphenols that differ from one another. As a result, they have different properties. In order to take this into account, it is useful to record at least one reference spectrum for each type of polyphenol and to compare a spectrum of the liquid that is to be stabilized with at least one of the reference spectra. From the resulting comparison data, it becomes possible to estimate the quantity of the type of polyphenol corresponding to each reference spectrum.

For the reference spectrum, a type of polyphenol can be drawn upon that, based on empirical and/or scientific principles, is one that is expected to exist in the liquid that is to be stabilized. As a result of this, a more precise evaluation of polyphenol content is possible. This is because the spectrum of the liquid and the reference spectrum can be compared in a defined and restricted spectral range in which a spectral line pattern that is characteristic for the type of polyphenol would be present.

Some practices include recording plural spectra while the method is being carried out, i.e. as the concentration of polyphenol changes. These plural spectra are then available for comparative evaluation and analysis in the form of a calibration series.

Among these practices are those that include comparing the liquid's spectrum with that of concentration-dependent data from the reference spectrum. Based on the liquid's spectrum and at least in part on the basis of the data from the reference spectrum prepared as a calibration series, the process reads off a quantity or concentration of polyphenol in the liquid.

Further practices include selecting the polyphenol from which a reference spectrum is recorded or provided and that is therefore drawn on for comparative analysis. Such selection can be made on the basis of empirical data or with the aid of values drawn from practical experience. Such practices include selecting a specific type of polyphenol that is found in the liquid that is to be stabilized. Such a selection can be based on experience or scientific evidence.

A polyphenol that is expected to always be present and/or that is present in the highest amount or in the highest proportion in the liquid to be stabilized is designated as the “lead polyphenol” for this liquid. From comparison analyses of the liquid's spectrum and of the lead polyphenol's spectrum, i.e., the reference spectrum, it is possible to determine the quantity of the lead polyphenol.

It is also possible for there to be more than one lead polyphenol. Polyphenols that are expected to always be present and/or that is present in the highest amounts or in the highest proportions in the liquid to be stabilized are designated as the “lead polyphenols” for this liquid. From comparison analyses of the liquid's spectrum and of the lead polyphenols' spectra, i.e., the reference spectra, it is possible to determine the quantities of the lead polyphenols.

In some cases, the liquid contains two or more types of polyphenol. In such cases, the method includes determining the quantity of total polyphenol contained in the liquid. This is carried out by first determining a quantity of the lead polyphenol. The method then includes using the quantity thus determined and suitable evaluation operations to calculate the quantity of the total polyphenol contained in the liquid.

In some cases, the liquid contains two or more types of polyphenol where the amount of total polyphenol is determined. In such cases, the method includes estimating a quantity of a guide polyphenol. Based on this estimate, and using a quantity of a lead polyphenol, the method includes carrying out evaluation operations to estimate the total amount of polyphenol in the liquid.

In those practices in which the liquid to be stabilized has two or more types of polyphenol, the method includes selecting a lead polyphenol for comparative analysis. It is particularly useful to select the lead polyphenol based on empirical data, published data, or with the aid of data arrived at from experience and to then estimate the amount of this lead polyphenol in the liquid that is to be stabilized based at least in part on such data. Once this has been done, the amount of the lead polyphenol is converted to a quantity for the total polyphenol in the liquid. This conversion is based on further empirical and scientific data or from experience obtained from actual practice.

It is often difficult to come by references in the literature to absolute quantities of polyphenols contained in a liquid that is to be stabilized or to concentrations of the total polyphenols contained in a liquid which is to be stabilized. Nevertheless, the types of polyphenols contained and the approximate proportional distribution or the approximate ratios of specific types of polyphenols to one another in a predefined liquid are known. The method exploits this known data to infer, from the quantity of the lead polyphenol, the total quantity of polyphenols contained in the liquid to be stabilized.

Some practices include determining the total polyphenol quantity based at least in part on quantities of more than two lead polyphenols. Such practices include determining the total polyphenol quantity based at least in part on quantities of two lead polyphenols, determining the total polyphenol quantity based at least in part on quantities of three lead polyphenols, and determining the total polyphenol quantity based at least in part on quantities of more than three lead polyphenols.

The determination includes obtaining reference spectra from the lead polyphenols. These spectra are either recorded or provided. In either case, they are made available for comparative evaluation. From the comparison, the method proceeds to estimate the quantity of total polyphenol contained in the liquid. This estimate is based at least in part on empirical data relating to the proportional distribution of polyphenols in specific liquids.

Some practices include selecting the liquid to be beer. In such cases, one or more lead polyphenols are selected from a group of flavonoids, preferably from a group of chalkons and/or flavons and/or anthocyanides, from a group of proanthocyanides, and for very particular preference from a group of dimer B-type proanthocyanides. Other practices include selecting one or more lead polyphenols from procyandin B3 or flavan-3-ol-dimer or prodelphiniden B3 or xanthohumol, or a desired mixture of procyandin B3 and/or flavan-3-ol-dimer and/or prodelphiniden B3 and/or xanthohumol.

The proportion of polyphenols contained in beer derives from the malt and from the hops used. Among these are hop polyphenols that are quite specific and that are present almost exclusively in hops. It is particularly useful to select one or more hop polyphenols as corresponding to one or more lead polyphenols when stabilizing beer or other hop-containing beverages such as ale.

Some practices include repeating the stabilization procedure at predetermined intervals of time. In the limit, as the intervals are reduced to zero, the stabilization procedure effectively becomes a continuous process. In such cases, the stabilization procedure can be integrated into an ongoing process, such as a process of beverage production and filling. Such cases can include repeating all method steps predetermined intervals of time such that stabilization is carried out as part of an inline process. The determination of the quantity of polyphenol in the liquid to be stabilized can therefore also be understood as an analytical step of an inline process, which is recurrently repeated during the inline process as a whole.

For example, beer that is to be stabilized is introduced and delivered from a mash unit or fermentation unit or pre-filtration unit, coming in a product stream into the supplying-and-feeding unit. From there, the beer flows through the measuring device, in which, for example using IR spectroscopy, the IR spectrum of the beer and, as appropriate, the IR reference spectrum is or was recorded. At the comparison-and-analysis unit, arranged upstream of the measuring device, the analyses and calculations for the determination of the quantity value of polyphenol in the beer run essentially parallel. After running through the measuring device, the product flow passes the metering unit, with the aid of which the stabilizing agent is added to the product flow in proportion to the quantity and as a function of the quantity value of polyphenol that has been determined. After the addition of the stabilizing agent, the mixture of beer and stabilizing agent is conveyed to a filter unit for the separation of the stabilizing agent, and the stabilized beer is then discharged through the discharge line and conveyed, for example, to a buffer container or tank or a filling system. All the steps are repeated at predetermined intervals of time, such that, over an entire production batch of the beer, the stabilizing takes place in a manner that remains consistent in degree and quality.

In carrying out the spectroscopic measurement, all methods are suitable provided that they permit the liquid being examined to be excited sufficiently for the formation of an analyzable spectrum. Thus, spectroscopic measurements within the scope of the method include those made in visible light, invisible light, infrared radiation, near-infrared radiation, and electromagnetic radiation with suitable wavelengths.

Some practices include selecting the stabilizing agent to be polyvinylpolypyrrolidon (PVPP). Other practice include selecting the stabilizing agent to be an absorber that can safely be used in foods or an enzyme.

Other practices include determining the quantity of substances other than polyphenols, such as impurities or contaminants, both qualitatively and quantitatively using similar methods provided that suitable reference substances and suitable measuring conditions and comparison and calculation operations are available and can be taken into account.

Practices include those in which estimating the quantity of the polyphenol contained in the liquid includes carrying out infrared spectroscopy. In some practices, infrared spectroscopy is carried out in the mid-infrared. In other practices, infrared spectroscopy is carried out in a range of wave numbers between 4000 waves per centimeter and 400 waves per centimeter. In other practices, infrared spectroscopy is carried out in the near-infrared. In other practices, infrared spectroscopy is carried out in a range of wave numbers 12500 waves per centimeter and 4000 waves per centimeter. In yet other practices, infrared spectroscopy is carried out over a band of wave numbers bounded by 50 waves per centimeter.

In another aspect, the invention feature an apparatus for stabilizing a liquid containing at least one type of polyphenol. Such an apparatus includes at least one supplying-and-feeding unit for supplying the liquid containing at least one type of polyphenol, at least one measuring device for recording at least one spectrum of the liquid, a comparison-and-analysis unit for comparing spectra and deriving from the comparison, using suitable comparison operations, an estimate of the quantity of polyphenol contained in the liquid, a metering unit for adding a specific quantity of a stabilizing agent to the liquid, the stabilizing agent being insoluble in the liquid, a filter unit for separating the stabilizing agent, which is at least partially loaded with polyphenol, and a discharge line for the discharge of the stabilized, which by this point has been depleted of the at least one type of polyphenol.

In some embodiments, the measuring device is in communication with the comparison-and-analysis unit.

In other embodiments, comparison-and-analysis unit is configured for recording, calculation, output, and storage of the raw data produced by the measuring device. Among these embodiments are those in which the comparison-and-analysis unit is configured for electronic image processing.

Yet other embodiments include a further measuring device for measuring a spectrum.

Still other embodiments feature a control unit that communicates with the comparison-and-analysis unit for controlling the metering unit.

Embodiments further include those in which the apparatus is configured for use with or for integration into a system for beverage production.

### BRIEF DESCRIPTION OF THE DRAWINGS

The invention is explained in greater detail hereinafter on the basis of exemplary embodiments in connection with the drawings, in which

FIG. 1 shows a device for stabilizing a liquid, and

FIG. 2 shows an alternative embodiment of a device for stabilizing a liquid.

### DETAILED DESCRIPTION

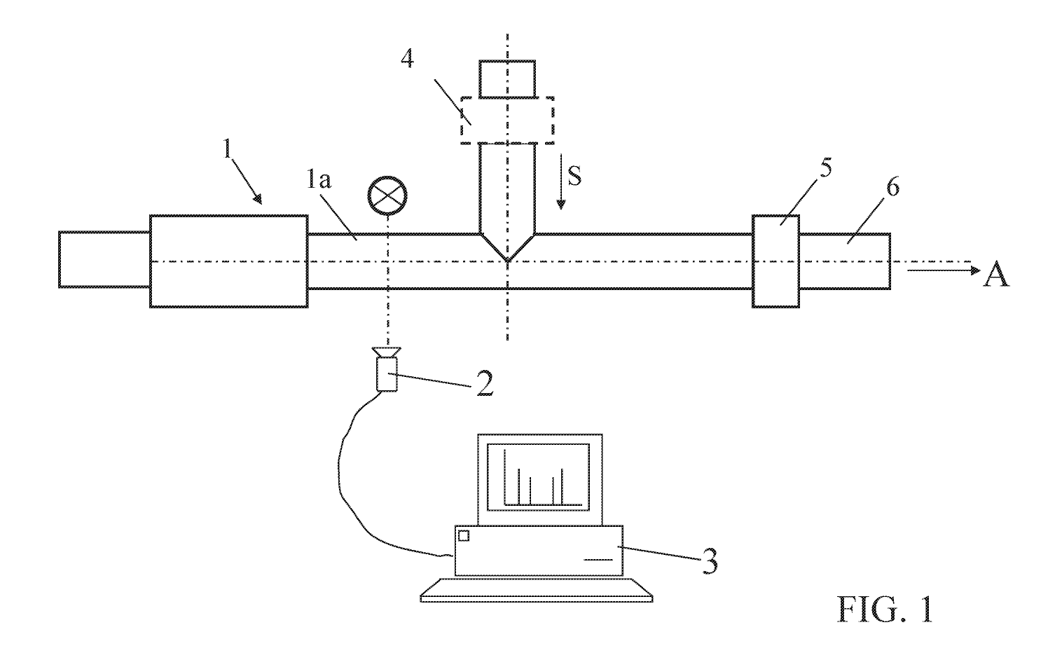

FIG. 1 shows an apparatus for carrying out a continuous process for stabilizing a liquid, such as beer. The steps of the method are integrated into the overall process of producing beer and filling it into containers.

The apparatus includes a feeding unit 1 that receives beer t delivered from a mash unit, a fermentation unit, or a pre-filtration unit into a supplying-and-feeding unit 1. The beer A flows past a measuring device 2 arranged in a light-permeable section 1a of the feeding unit 1.

A suitable measuring device 2 is a spectrometer, such as an infrared (IR) spectrometer. When an infrared light is passed through the light-permeable section 10, the measuring device 2 records the beer's IR spectrum for comparison with one or more IR reference spectra of one or more types of lead polyphenols.

In some embodiments, the infrared light radiates into the product flow with mid-infrared light having a wavelength range from 4000 waves per centimeter to 400 waves per centimeter. The beer absorbs certain wavelengths of the radiated light. What light remains enters the measuring device 2. A comparison-and-analysis unit 3, hereafter referred to more concisely as an “analysis unit,” receives light from the measuring device 2. The analysis unit 3 examines the light, and in particular, its spectral lines.

The analysis unit 3 carries out real-time comparison of the spectrum against one or more reference spectra corresponding to one or more types of lead polyphenols. As a result of this quantitative analysis, the analysis unit 3 estimates the amount of polyphenol in the beer.

After flowing past the measuring device 2, the product flow A passes through a metering unit 4. Under control of a control that is in communication with the analysis unit 3, the metering unit 4 stabilizing agent S into the beer A. A suitable stabilizing agent is polyvinylpolypyrrolidon (PVPP). The controller regulates the flow of stabilizing agent S based on the analysis unit's estimate of how much polyphenol is in the beer. This permits stabilizing agent S to be added to the beer A in an amount that is proportional to the polyphenol content of the beer A.

The mixture of beer A and stabilizing agent S flows to a filter unit 5. The dwell time, during which the beer A is in contact with the stabilizing agent S can be adjusted by either adjusting the flow rate or adjusting the distance between the filter unit 5 and the point at which the stabilizing agent S is added.

The filter unit 5 separates the stabilizing agent S from the beer A. The stabilized beer A is then discharged through a discharge line 6 and conveyed, for example, to a buffer container, a tank, or a filling system.

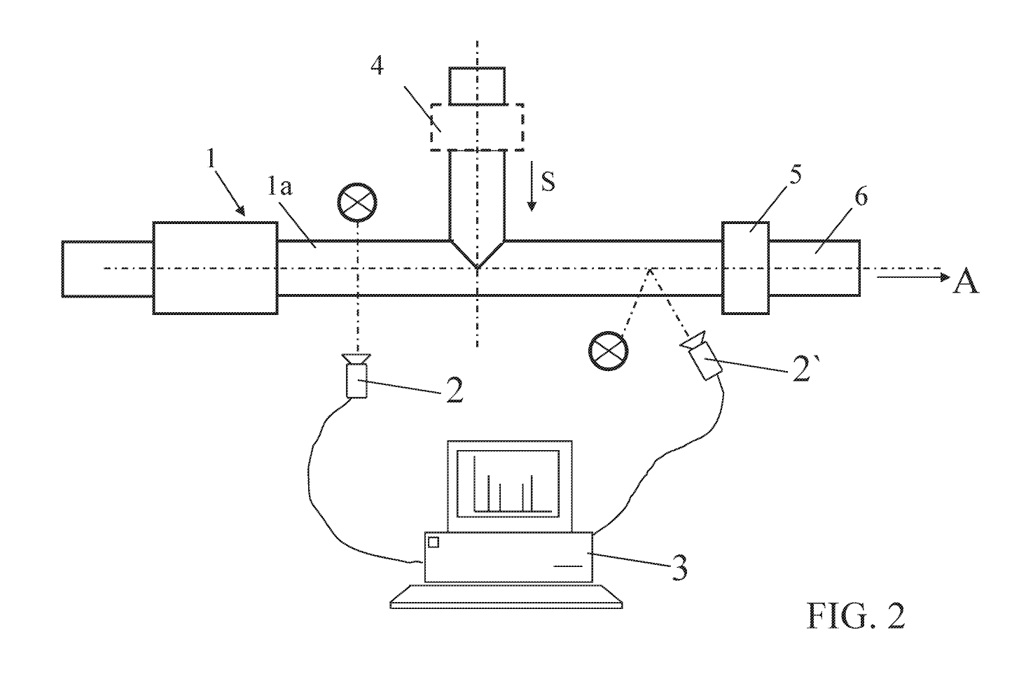

An alternative embodiment, shown in FIG. 2, features a first measuring device 2 and a second measuring device 2′ downstream of the first measuring device 2 and a metering unit 4. The first and second measuring devices 2, 2′ record corresponding first and second IR spectra of the beer A.

In the embodiment of FIG. 2, the analysis unit 3 receives both the first and second IR spectra in the manner already described in connection with the first embodiment.

By evaluation of the data obtained from the second IR spectrum of the beer, it becomes possible to monitor the degree of stabilization. If the desired degree of stabilization is not attained, it is possible to adjust the dwell time, for example by adjusting flow rate, or to adjust the amount of stabilizing agent S that is introduced at the metering unit 4.

Having described the invention, and a preferred embodiment thereof, what is claimed as new and secured by letters patent is:

1-18. (canceled)

19. A process comprising stabilizing a liquid that contains a first type of polyphenol, wherein stabilizing said liquid comprising supplying said liquid through a supplying-and-feeding unit, using a measuring device, measuring a spectrum of said liquid, using a comparison-and-analysis unit, comparing said spectrum with a reference spectrum, based at least in part on said comparison, estimating an amount of said first type of polyphenol, adding a specified quantity of a stabilizing agent to said liquid, feeding said stabilizing agent and said liquid to a filter unit, at said filter unit, separating said stabilizing agent from said liquid, and discharging said liquid, wherein said stabilizing agent is insoluble in said liquid, wherein said specified quantity is based on said amount, wherein said stabilizing agent is configured to bind to said first type of polyphenol, wherein said stabilizing agent that is removed has been partially loaded with polyphenol, and wherein said liquid that is discharged is a stabilized liquid that has been depleted of at least one type of polyphenol.

20. The process of claim 19, further comprising, prior to comparing said spectrum with a reference spectrum, using said measuring device to ascertain said reference spectrum.

21. The process of claim 19, further comprising, prior to comparing said spectrum with a reference spectrum, obtaining a reference spectrum of said first type of polyphenol, and wherein comparing said spectrum with a reference spectrum comprises, using said comparison-and-analysis unit, estimating an amount of said first type of polyphenol contained in said liquid, wherein estimating comprises comparing said spectrum with said reference spectrum, and, from said comparison, calculating a value indicative of a quantity of said first type of polyphenol contained in said liquid.

22. The process of claim 19, wherein supplying said liquid through a supplying-and-feeding unit comprises supplying a liquid that contains plural types of polyphenols, and wherein comparing said spectrum with a reference spectrum, based on said comparison and estimating an amount of said first type of polyphenol comprises determining a total quantity of polyphenols in said liquid.

23. The process of claim 22, wherein comparing said spectrum with a reference spectrum comprises, using said comparison-and-analysis unit, from said comparison of said spectrum of said liquid with said at least one IR reference spectrum by comparison operations, estimating a quantity of at least one type of a lead polyphenol in said liquid, and, from said estimate, estimating a quantity of total polyphenols contained in said liquid.

24. The process of claim 23, wherein estimating a quantity of total polyphenols in said liquid comprises estimating quantities of plural types of lead polyphenols in said liquid, and, based at least in part on said quantities, estimating said quantity of total polyphenols contained in said liquid.

25. The process of claim 19, wherein supplying said liquid through a supplying-and-feeding unit comprises supplying a beverage containing polyphenols.

26. The process of claim 24, further comprising selecting said plural types of lead polyphenols from the group of flavonoids, chalkons, flavons, anthocyanides, proanthocyanides, and dimer B-type proanthocyanides.

27. The process of claim 24, further comprising selecting said plural types of lead polyphenols from the group consisting of procyandin B3, flavan-3-ol-dimer, prodelphiniden B3, xanthohumol, and a mixture, wherein said mixture is a mixture of substances selected from the group consisting of procyandin B3, flavan-3-ol-dimer, prodelphiniden B3, and xanthohumol.

28. The process of claim 19, further comprising repeating stabilizing said liquid at predetermined intervals of time.

29. The process of claim 19, further comprising selecting said stabilizing agent to comprise polyvinylpolypyrrolidone.

30. The process of claim 19, wherein measuring a spectrum of said liquid comprises measuring an IR spectrum of said liquid.

31. The process of claim 19, wherein measuring an IR spectrum comprises measuring a spectrum having mid-infrared wavelengths.

32. The process of claim 19, wherein measuring an IR spectrum comprises measuring a spectrum having near-infrared wavelengths.

33. The process of claim 19, wherein measuring an IR spectrum comprises measuring a spectrum between 4000 waves per centimeter and 400 waves per centimeter.

34. The process of claim 19, wherein measuring an IR spectrum comprises measuring a spectrum having a wavelength between 12500 waves per centimeter to 4000 waves per centimeter.

35. The process of claim 19, wherein measuring a spectrum of said liquid comprises measuring said spectrum within a wavelength range having an upper bound of 50 waves per centimeter.

36. The process of claim 19, wherein measuring a spectrum of said liquid comprises measuring a visible light spectrum.

37. An apparatus for specifically designed for carrying out a stabilization process as recited in claim 19, wherein said apparatus comprises a supplying-and-feeding unit, a first spectroscope, a comparison-and-analysis unit, a metering unit, a filter, and a discharge line, wherein said supplying-and-feeding unit is configured for providing liquid containing at least one type of polyphenol, wherein said first spectroscope is configured for ascertaining at least one spectrum of said liquid, wherein said comparison-and-analysis unit is configured for comparing spectra and, based on said comparison, estimating a quantity of polyphenol contained in said liquid, wherein said metering unit is configured for adding a specific amount of a stabilizing agent to said liquid, said stabilizing agent being insoluble in said liquid, wherein said filter is configure for separating, from said liquid, stabilizing agent that has been at least partially loaded with polyphenol, and wherein said discharge line is configured for discharging said stabilized liquid, said stabilized liquid having been depleted of at least one type of polyphenol.

38. The apparatus of claim 37, further comprising a control unit configured for communicating connection to said comparison-and-analysis unit and for controlling said metering unit.

39. The apparatus of claim 37, further comprising a second spectroscope.

40. The apparatus of claim 37, further comprising selecting said liquid to be a beverage.
